(19) United States
(12) Patent Application Publication
(10) Pub. No.: US 2026/0118327 A1
(43) Pub. Date: Apr. 30, 2026

(54) EXHALED BREATH DETECTION APPARATUS, CALIBRATION INFORMATION GENERATION APPARATUS, AND NON-TRANSITORY COMPUTER-READABLE MEDIUM

(71) Applicant: Asahi Kasei Microdevices Corporation, Tokyo (JP)

(72) Inventors: Yuta TAKAGI, Tokyo (JP); Keiichiro KUWATA, Tokyo (JP)

(21) Appl. No.: 19/367,754

(22) Filed: Oct. 23, 2025

(30) Foreign Application Priority Data
Oct. 31, 2024 (JP) 2024-192339
Sep. 25, 2025 (JP) 2025-159365

(51) Int. Cl. G01N 33/00; B60K 28/06; G01N 1/22; G01N 33/497

(52) U.S. Cl. CPC G01N 33/0006; B60K 28/06; G01N 1/2226; G01N 1/2247; G01N 1/2273; G01N 33/004; G01N 33/4972; G01N 2001/2244

(57) ABSTRACT

Provided is an exhaled breath detection apparatus comprising: a component detection unit which obtains, through detection, detection information indicating a concentration of alcohol and a concentration of carbon dioxide contained in exhaled breath; a calibration information generation unit which generates calibration information for calibrating a concentration of the carbon dioxide, based on the detection information of the carbon dioxide obtained through multiple detections by the component detection unit; and a result correction unit which corrects a detection result of the alcohol based on a calibrated concentration of the carbon dioxide obtained through calibration with the calibration information.

The contents of the following patent application(s) are incorporated herein by reference:

No.2024-192339 filed in JP on October 31, 2024 and

No. 2025-159365 filed in JP on September 25, 2025.

### BACKGROUND

### 1. TECHNICAL FIELD

The present invention relates to an exhaled breath detection apparatus, a calibration information generation apparatus, and a non-transitory computer-readable medium.

### 2. RELATED ART

Patent document 1 discloses a method for evaluating blood concentration that is “based on measurement of both alcohol and carbon dioxide concentrations in exhaled breath that has flowed out from a subject's mouth and has been diluted by ambient air” (paragraph 0046).

Patent Document 2 discloses an exhaled breath collection method in which “a mouthpiece, previously considered essential for exhaled breath collection apparatuses, is no longer required” (paragraph 0030).

### RELATED ART DOCUMENTS

### Patent Documents

Patent Document 1: Japanese Patent No. 7827087

Patent Document 2: Japanese Patent No. 5502269

### BRIEF DESCRIPTION OF THE DRAWINGS

FIG. 1 is a schematic diagram showing an example of a mobile body 200 equipped with an exhaled breath detection apparatus 100 according to an embodiment of the present invention.

FIG. 2 is a block diagram showing an example configuration of the exhaled breath detection apparatus 100.

FIG. 3 shows an example of temporal variation of a carbon dioxide concentration inside an operator’s cabin 240.

FIG. 4 is a block diagram showing another example configuration of the exhaled breath detection apparatus 100.

FIG. 5 is a schematic diagram illustrating a state of an air supply unit 104.

FIG. 6 is a schematic diagram illustrating a state of the air supply unit 104.

FIG. 7 is a flowchart illustrating an example operation of the exhaled breath detection apparatus 100 illustrated in FIG. 4.

FIG. 8 shows another example configuration of the exhaled breath detection apparatus 100.

FIG. 9 shows an example configuration of an intake port 102, the air supply unit 104, a detection housing 106, and a component detection unit 110.

FIG. 10, shows another example configuration of the intake port 102, the air supply unit 104, the detection housing 106, and the component detection unit 110.

FIG. 11 shows an example of a computer 1200 in which a plurality of aspects of the present invention may be entirely or partially embodied.

### DESCRIPTION OF EXEMPLARY EMBODIMENTS

Hereinafter, the present invention will be described through embodiments of the invention, but the following embodiments do not limit the invention according to the claims. In addition, not all combinations of features described in the embodiments are necessarily essential to a solution of the invention.

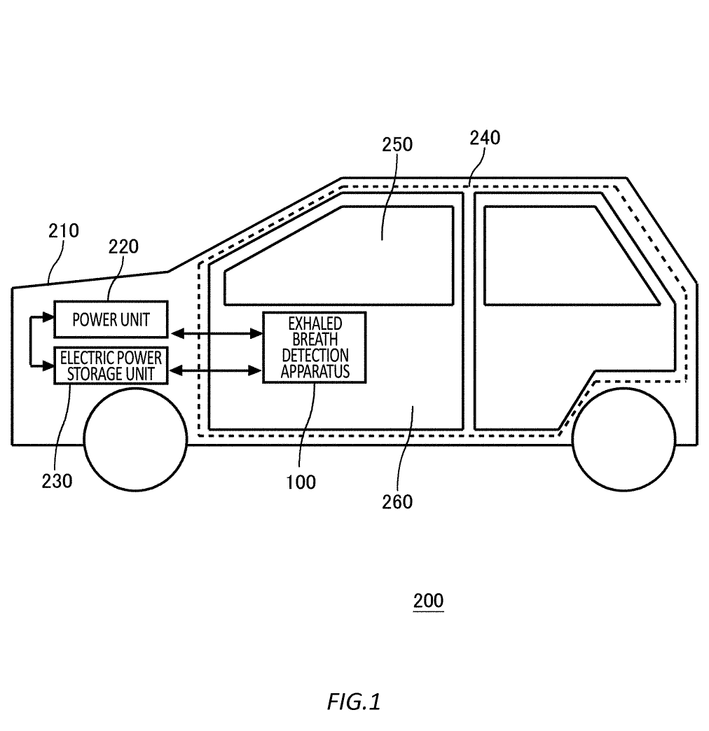

FIG. 1 is a schematic diagram showing an example of a mobile body 200 equipped with an exhaled breath detection apparatus 100 according to an embodiment of the present invention. The mobile body 200 is, for example, an automobile, but is not limited thereto. The mobile body 200 may be a ground mobile body such as a vehicle which moves on the ground, may be an airborne mobile body such as a flying object which flies through air, may be a waterborne mobile body such as a vessel which moves on the water, may be an underwater mobile body such as a submersible which moves underwater, or may be a mobile body which moves in another location.

The mobile body 200 includes an operator’s cabin 240 for boarding by an operator who operates the mobile body 200. The operator’s cabin 240 may include a space for boarding by a passenger other than an operator. The operator’s cabin 240 is, for example, a space in which equipment for operation of the mobile body 200, such as a steering wheel in an automobile, is installed. The operator’s cabin 240 in the present example is a space surrounded by a mobile body housing 210 of the mobile body 200. The mobile body housing 210 includes, for example, a body portion of an automobile. The mobile body housing 210 may include at least one of one or more windows 250 or one or more doors 260. The window 250 and the door 260 may be openable and closable between the operator’s cabin 240 and an exterior space. The mobile body housing 210 may include, in addition to the window 250 and the door 260, a portion which is openable and closable.

The exhaled breath detection apparatus 100 detects alcohol contained in exhaled breath in the operator’s cabin 240 of the mobile body 200. The exhaled breath detection apparatus 100 detects alcohol contained in exhaled breath of an operator by measuring air in the operator’s cabin 240. A part or a whole of the exhaled breath detection apparatus 100 is provided in the operator’s cabin 240. The exhaled breath detection apparatus 100 may detect a concentration of alcohol (ppm). The exhaled breath detection apparatus 100 may determine whether or not a detected concentration of alcohol is within an allowable range. The exhaled breath detection apparatus 100 may prohibit an operator from operating the mobile body 200 when a detected concentration of alcohol exceeds an allowable range, may restrict speed and movement range of the mobile body 200, or may restrict human operation and switch to an autonomous driving mode. The exhaled breath detection apparatus 100 may estimate a blood alcohol concentration of an operator based on an alcohol concentration contained in detected exhaled breath.

If a detected concentration of alcohol exceeds an allowable range, the exhaled breath detection apparatus 100 may present an alert including this information to an operator, may record the detected alcohol concentration, time, the operator, and a location, and may present a notification to the operator indicating that a concentration of alcohol is to be remeasured. The exhaled breath detection apparatus 100 may acquire location information of the mobile body 200 and may change an allowable range of an alcohol concentration based on the acquired location information. For example, in Japan, an alcohol concentration in exhaled breath of 0.15 mg/L or higher is considered driving under the influence, whereas in Germany, an alcohol concentration in exhaled breath of 0.25 mg/L or higher constitutes a legal violation, therefore, the exhaled breath detection apparatus 100 may change an allowable range of an alcohol concentration based on location information of the mobile body 200.

The mobile body 200 in the present example includes a power unit 220 and an electric power storage unit 230. The power unit 220 generates power to move the mobile body 200. The power unit 220 may be, for example, an internal combustion engine such as an engine, which generates power by combusting fuel. The power unit 220 may be, for example, an electric motor such as a motor, which rotates according to electric power. The exhaled breath detection apparatus 100 may cause power generation by the power unit 220 to be stopped when a detected concentration of alcohol exceeds an allowable range. When the power unit 220 is an electric motor such as a motor, the power unit 220 may include a fuel cell as an electric power source, and the exhaled breath detection apparatus 100 may stop operation of the fuel cell when a detected concentration of alcohol exceeds an allowable range.

The electric power storage unit 230 stores electric power and supplies electric power to equipment of the mobile body 200. The electric power storage unit 230 may supply electric power to the power unit 220, may supply electric power to the exhaled breath detection apparatus 100, or may supply electric power to other equipment such as an air conditioner that adjusts a temperature in the operator’s cabin 240 and a display unit that displays information, or the like. The exhaled breath detection apparatus 100 may cause electric power supply from the electric power storage unit 230 to be stopped when a detected concentration of alcohol exceeds an allowable range.

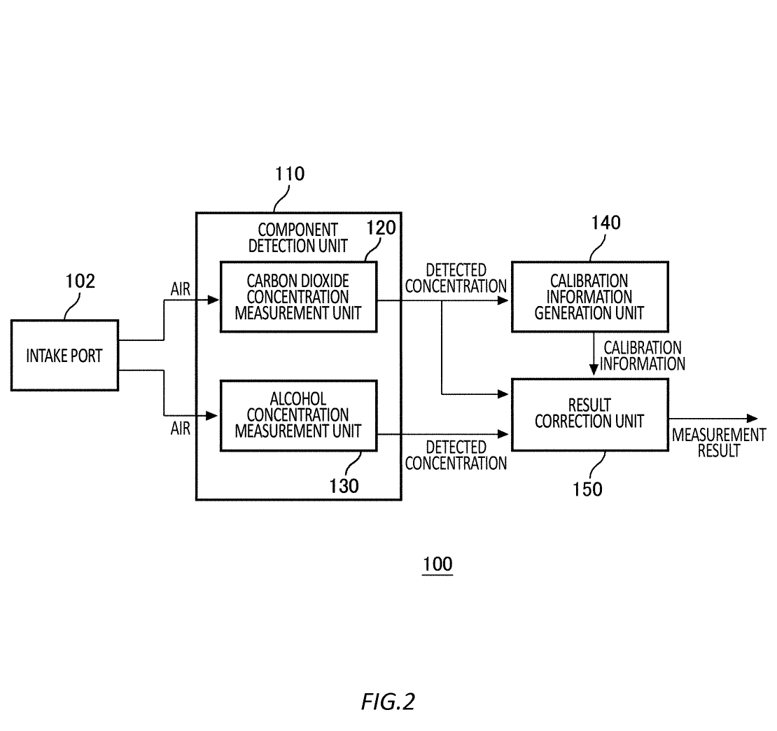

FIG. 2 is a block diagram showing an example configuration of the exhaled breath detection apparatus 100. The exhaled breath detection apparatus 100 includes a component detection unit 110, a calibration information generation unit 140, and a result correction unit 150. The exhaled breath detection apparatus 100 may further include an intake port 102.

The component detection unit 110 obtains, through detection, detection information indicating a concentration of alcohol and a concentration of carbon dioxide contained in exhaled breath. The component detection unit 110 may obtain, through detection, detection information indicating a concentration of alcohol and detection information indicating a concentration of carbon dioxide. Each piece of detection information is a signal whose value varies according to a magnitude of a concentration of each target component (alcohol and carbon dioxide in the present example) contained in exhaled breath. For example, detection information is a signal whose value corresponds to intensity of light that has passed through gas containing exhaled breath, at wavelengths corresponding to each target component. The intensity of the light is attenuated according to concentration of each target component contained in exhaled breath. Detection information may be a signal obtained by converting the signal of the light into an electric signal, or may be a signal obtained by performing predetermined signal processing on the electric signal. Detection information may include concentration values themselves for each target component.

In the present example, an apparatus having the component detection unit 110 which obtains, through detection, detection information indicating a concentration of alcohol and a concentration of carbon dioxide contained in exhaled breath is described as the exhaled breath detection apparatus 100 which detects alcohol contained in exhaled breath. In another example, the component detection unit 110 may be configured to obtain, through detection, detection information indicating a concentration of alcohol and a concentration of oxygen. The exhaled breath detection apparatus 100 may, in calculating a degree of dilution, use a concentration of oxygen in place of a concentration of carbon dioxide as in respective example embodiments described in the present specification. In this case, carbon dioxide in the present specification may be read as oxygen. For example, the exhaled breath detection apparatus 100 may assume that an average oxygen concentration in atmosphere is 21%, perform calibration based on this average oxygen concentration, and calculate a dilution rate of exhaled breath based on a calibrated concentration of oxygen. In yet another example, the component detection unit 110 may obtain, through detection, detection information indicating a concentration of alcohol, a concentration of carbon dioxide, and a concentration of oxygen contained in exhaled breath. In this case, the component detection unit 110 includes an alcohol concentration measurement unit, a carbon dioxide concentration measurement unit, and an oxygen concentration measurement unit. The exhaled breath detection apparatus 100 may calculate a degree of dilution of exhaled breath based on a first degree of dilution calculated based on a concentration of carbon dioxide and a second degree of dilution calculated based on a concentration of oxygen. For example, the exhaled breath detection apparatus 100 may treat an average value of the first degree of dilution and the second degree of dilution as a degree of dilution of exhaled breath. By combining carbon dioxide concentration measurement and oxygen concentration measurement, a more accurate dilution rate of exhaled breath can be calculated.

The component detection unit 110 in the present example includes a carbon dioxide concentration measurement unit 120 and an alcohol concentration measurement unit 130. The carbon dioxide concentration measurement unit 120 outputs detection information corresponding to a concentration of carbon dioxide (ppm) contained in the air in the operator’s cabin 240 which is introduced into the component detection unit 110 via the intake port 102. The carbon dioxide concentration measurement unit 120 is, for example, a photoacoustic type sensor, a solid electrolyte type sensor, or a non-dispersive infrared absorption (NDIR) type sensor.

The alcohol concentration measurement unit 130 outputs detection information corresponding to a concentration of alcohol (ppm) contained in the air in the operator’s cabin 240 which is introduced into the component detection unit 110 via the intake port 102. The alcohol concentration measurement unit 130 may be, for example, an electrochemical (fuel cell) type sensor or may be a non-dispersive infrared absorption (NDIR) type sensor. An electrochemical type sensor detects, for example, an electric current generated by alcohol contained in air.

The calibration information generation unit 140 generates calibration information for calibrating a concentration of carbon dioxide based on detection information of carbon dioxide obtained through multiple detections by the component detection unit 110. Calibration information is information for converting a value of each piece of detection information into a concentration of each target component. Calibration information may be a calibration curve which indicates a relationship between a value of detection information and a concentration of a target component. When detection information includes concentration values themselves for each target component, calibration information may be information for correcting the concentration values in the detection information. In the present specification, a concentration calculated from a value of detection information by using calibration information may be referred to as a “calibrated concentration.”

For example, calibration information may include a gain value which is multiplied by a value of detection information to calculate a calibrated concentration, may include a function which calculates a calibrated concentration using a value of detection information as a variable, or may include a table in which a value of detection information is associated with a calibrated concentration.

An initial value of calibration information may be preset in the calibration information generation unit 140. The initial value of the calibration information may be set by a manufacturer, a user, or the like of the exhaled breath detection apparatus 100. The calibration information generation unit 140 may update calibration information based on detection information of carbon dioxide obtained through multiple detections by the component detection unit 110. In the present specification, updating of calibration information may be referred to as generation of calibration information.

Measurement accuracy of the carbon dioxide concentration measurement unit 120 may change over time. For example, in an NDIR type sensor, changes over time in characteristics of a light source, a light-guiding unit, or a light-receiving element may cause a deviation in a value of detection information generated by the light-receiving element, even when carbon dioxide of a same concentration is measured. As a result, a deviation may be caused in a calibrated concentration obtained through calibration even when carbon dioxide of the same concentration is measured. The calibration information generation unit 140 generates or updates calibration information so that a deviation in a calibrated concentration obtained through calibration remains small, even when a value of detection information for carbon dioxide of the same concentration changes over time.

If a difference between calibrated concentrations for predetermined detection information before and after updating calibration information exceeds a reference value, the exhaled breath detection apparatus 100 may perform exception processing because there is a possibility that the exhaled breath detection apparatus 100 is not operating normally. The reference value may be 500 ppm, may be 100 ppm, or may be any other value. The exception processing may be correction of calibration information so that the difference becomes equal to or less than a reference value, may be reacquisition of calibration information, or may be notifying an operator of an abnormal state.

The calibration information generation unit 140 may extract, from among detection information of carbon dioxide obtained through multiple detections, the detection information whose corresponding concentration is the lowest. In the present specification, a relative magnitude relationship between corresponding concentrations may be described as a relative magnitude relationship between detection information. For example, among a plurality of pieces of detection information, detection information whose corresponding concentration is the lowest may be referred to as minimum detection information. The calibration information generation unit 140 may adjust the aforementioned calibration information so that the minimum detection information is converted into a preset reference concentration. Adjusting calibration information may be adjusting the aforementioned gain value, may be adjusting each coefficient of a function, or may be updating a table. For example, the calibration information generation unit 140 may calculate the gain value by dividing a reference concentration by a concentration corresponding to the minimum detection information. For example, the reference concentration may be equivalent to an average carbon dioxide concentration in outside air. The reference concentration may be 400 ppm or may be other value.

The calibration information generation unit 140 may generate calibration information by using a plurality of pieces of detection information measured within a most recent reference period among previously measured detection information. The reference period may be preset by a manufacturer or a user of the exhaled breath detection apparatus 100. The reference period may be one week or longer, may be 2 weeks or longer, or may be one month or longer. The reference period may be equal to or shorter than 6 months, or may be equal to or shorter than 2 months.

A carbon dioxide concentration in the operator’s cabin 240 fluctuates due to exhaled breath of an operator or a passenger. Meanwhile, a carbon dioxide concentration in the operator’s cabin 240 is never lower than a carbon dioxide concentration in outside air. Accordingly, it can be estimated that detection information having smaller values resulted from measurements taken in a state closer to a carbon dioxide concentration in outside air. Therefore, by adjusting calibration information so that detection information with a minimum value among a plurality of pieces of detection information is converted into a reference concentration, calibration information with relatively high accuracy can be generated.

A number of a plurality of pieces of detection information used to generate calibration information may be 10 or more, may be 100 or more, or may be 1000 or more. A method for generating calibration information is not limited to this method. The calibration information generation unit 140 may use a statistical value obtained from a plurality of pieces of detection information, selected in ascending order of their values, from among the plurality of pieces of detection information. The statistical value refers to information obtained by performing statistical processing on a plurality of pieces of detection information, or the statistical processing may be generating at least one of an average value, a maximum value, a minimum value, a variance, a moment, or a histogram.

The result correction unit 150 corrects a detection result of alcohol based on a calibrated concentration of carbon dioxide obtained through calibration with calibration information. The detection result of alcohol is, for example, a concentration of alcohol. The result correction unit 150 corrects a concentration of alcohol in a measurement target with a calibrated concentration of carbon dioxide measured concurrently with the concentration of the alcohol.

For example, the result correction unit 150 calculates a degree of dilution of air which has reached the component detection unit 110, based on a calibrated concentration of carbon dioxide. A degree of dilution is an index indicating how much exhaled breath of an operator has been diluted until it reaches the component detection unit 110 from an operator. A degree of dilution may be a value obtained by dividing a preset standard concentration of carbon dioxide by a calibrated concentration of carbon dioxide. For the standard concentration of carbon dioxide, an average value of concentrations of carbon dioxide contained in exhaled breath of adults may be used, or it may be a value actually measured from an exhaled breath of an operator. The standard concentration of carbon dioxide is, for example, a value in a range from 1% to 9%. The standard concentration of carbon dioxide may be, for example, 3%. The standard concentration of carbon dioxide may be set by a manufacturer or a user of the exhaled breath detection apparatus 100. For example, when the standard concentration of carbon dioxide is 3% and a calibrated concentration of carbon dioxide is 1000 ppm, a degree of dilution may be 30.

The result correction unit 150 may calculate a corrected concentration of alcohol by multiplying the concentration of alcohol by the aforementioned degree of dilution. For example, when a degree of dilution is calculated as 150 times based on a calibrated concentration of carbon dioxide, the result correction unit 150 may calculate a corrected concentration of alcohol by multiplying the concentration of alcohol by 150. In this way, a concentration of alcohol contained in exhaled breath of an operator can be estimated. In another example, the result correction unit 150 may correct a threshold concentration for comparison with a concentration of alcohol, based on a degree of dilution. For example, when a degree of dilution is 150 times, the result correction unit 150 may correct a detection result of alcohol by dividing the threshold concentration by 150.

The exhaled breath detection apparatus 100 in the present example calculates a degree of dilution of air measured by the component detection unit 110 relative to exhaled breath of an operator based on a carbon dioxide concentration, and corrects a detection result of alcohol. As such, exhaled breath of an operator may not be directly blown into the component detection unit 110. The exhaled breath detection apparatus 100 in the present example can measure an alcohol concentration of an operator even in a state where the operator has no intention of measuring the alcohol concentration. Then, since calibration information for a carbon dioxide concentration is generated based on carbon dioxide concentrations (detection information in the present example) obtained through multiple measurements inside the operator’s cabin 240, changes over time in characteristics of the component detection unit 110 can be corrected, enabling a degree of dilution to be calculated with high accuracy and an alcohol concentration to be measured with high accuracy.

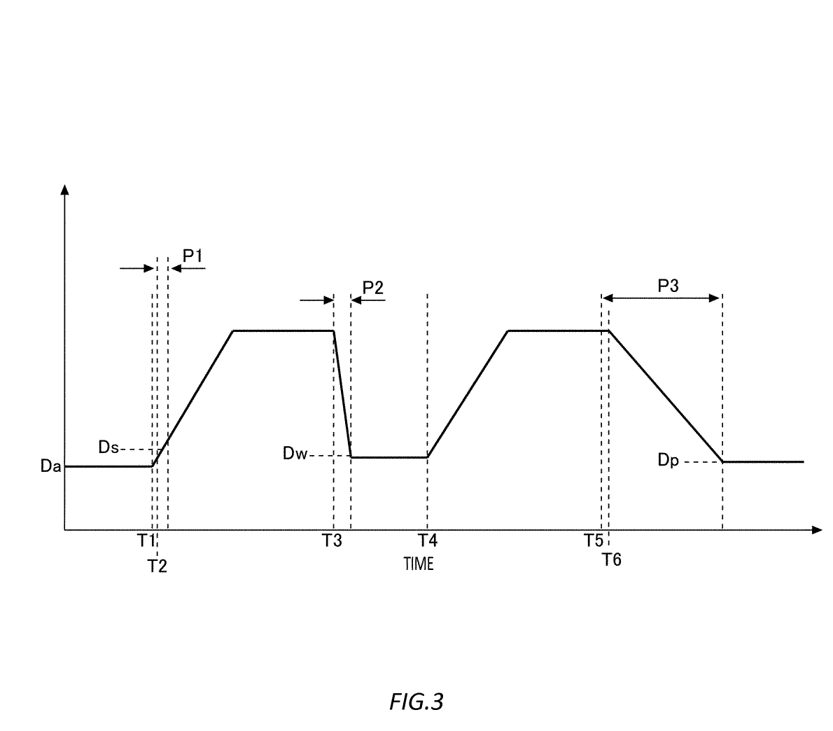

FIG. 3 shows an example of temporal variation of a carbon dioxide concentration inside the operator’s cabin 240. The carbon dioxide concentration in FIG. 3, is measured by the component detection unit 110. Furthermore, a carbon dioxide concentration in outside air is defined as Da.

When a sufficient amount of time elapses after an operator or the like leaves the operator’s cabin 240, the carbon dioxide concentration inside the operator’s cabin 240 becomes approximately equal to the outside air concentration Da. At time T1 in this state, when an operator enters the operator’s cabin 240, the carbon dioxide concentration gradually increases due to exhaled breath of the operator or the like.

At time T2, the component detection unit 110 is started and enters a state capable of measuring the carbon dioxide concentration inside the operator’s cabin 240. The time T2 may be a time when electric power supply to the component detection unit 110 is started. The time T2 may be simultaneous with the time T1. Furthermore, if the component detection unit 110 can be started by remote operation or the like without an operator entering the operator’s cabin 240, the time T2 may be earlier than the time T1. At the time T2, it is sufficient that the carbon dioxide concentration measurement unit 120 is started, and the alcohol concentration measurement unit 130 may be started after the time T2.

An operator who enters the operator’s cabin 240 may start up at least a portion of equipment of the mobile body 200 at the time T2. The operator may start up the power unit 220 of the mobile body 200 at the time T2. In this case, the mobile body 200 may start supplying energy such as fuel or electric power to the power unit 220 at the time T2. In the present example, upon startup of the power unit 220, other equipment of the mobile body 200, such as the component detection unit 110, is started. In another example, the operator may start up the component detection unit 110 at the time T2 without starting up the power unit 220.

The component detection unit 110 may detect a carbon dioxide concentration in the operator’s cabin 240 within a preset period P1 starting at the time T2. In the present specification, obtaining detection information through detection may be treated as detecting a concentration. That is, when detecting a concentration is referred to in the present specification, it means detecting corresponding detection information. The component detection unit 110 may obtain a carbon dioxide concentration through multiple detections within the period P1. In the present specification, a carbon dioxide concentration detected within the period P1 may be referred to as a startup-time concentration Ds. Further, in the present specification, detection information corresponding to the startup-time concentration is referred to as startup-time detection information. For example, the period P1 may be within one minute, may be within 30 seconds, or may be within 10 seconds. The component detection unit 110 may, once it reaches a state where it can stably detect a carbon dioxide concentration, detect the startup-time concentration Ds. For example, the component detection unit 110 may detect the startup-time concentration Ds on condition that a temperature of the carbon dioxide concentration measurement unit 120 falls within a predetermined set range.

The component detection unit 110 may detect the startup-time concentration Ds each time the power unit 220 or the component detection unit 110 is started. The calibration information generation unit 140 may generate calibration information by using the startup-time concentration Ds (or startup-time detection information) detected within the period P1. The calibration information generation unit 140 may generate calibration information by using a plurality of the startup-time concentrations Ds detected within a preset calibration target period. As described above, the calibration information generation unit 140 may generate calibration information with the lowest startup-time concentration Ds from among the plurality of the startup-time concentrations Ds. Since the startup-time concentration Ds is presumed to be relatively close to the outside air carbon dioxide concentration Da, calibration information can be generated with high accuracy by using the startup-time concentration Ds. The calibration information generation unit 140 may estimate the outside air concentration Da from changes over time in carbon dioxide concentrations obtained through multiple detections within the period P1, and may generate calibration information based on the estimated outside air concentration Da. For example, the calibration information generation unit 140 may estimate the outside air concentration Da, to which a carbon dioxide concentration converges, by approximating carbon dioxide concentrations obtained through multiple detections with a function of a preset order. The calibration information generation unit 140 may update calibration information so that a value of detection information corresponding to the estimated outside air concentration Da is converted into a reference concentration.

The calibration information generation unit 140 may select the startup-time concentration Ds (or startup-time detection information) to be used for generating calibration information, based on a temperature of the component detection unit 110 at startup of the component detection unit 110. For example, for each of the periods P1, the calibration information generation unit 140 may use the startup-time concentration Ds, acquired during the period P1, for generating calibration information, on condition that a temperature of the component detection unit 110 at startup is within a preset temperature range. In this way, influence of temperature variations in the component detection unit 110 at a time of measuring the startup-time concentration Ds, as well as influence of temperature characteristics of the component detection unit 110, can be reduced. The temperature range may allow the component detection unit 110 to detect the startup-time concentration Ds with high accuracy. The temperature range may be, for example, 10°C or higher and 30°C or lower. In another example, the calibration information generation unit 140 may correct each of the startup-time concentrations Ds based on a temperature of the component detection unit 110 at a time when the startup-time concentration Ds was measured. A relationship between the startup-time concentration Ds and a temperature is determined according to characteristics of the component detection unit 110. The relationship may be measured in advance by a manufacturer, a user, or the like of the component detection unit 110. The calibration information generation unit 140 may use a temperature inside the operator’s cabin 240 as a temperature of the component detection unit 110. The mobile body 200 may be provided with a temperature sensor which detects a temperature of the component detection unit 110 or a temperature inside the operator’s cabin 240. Furthermore, the component detection unit 110 may include a temperature adjustment unit for adjusting a temperature of the component detection unit 110. For example, by maintaining a temperature of the component detection unit 110 at 70°C, the temperature adjustment unit can reduce degradation in detection performance due to temperature characteristics or condensation of the component detection unit 110. The temperature adjustment unit may be a heater or a Peltier element, and may further include a thermometer or a control unit (for example, a microcontroller) for feedback control.

The calibration information generation unit 140 may select the startup-time concentration Ds (or startup-time detection information) to be used for generating calibration information, based on an external temperature of the mobile body 200 at startup of the component detection unit 110. For example, for each of the periods P1, the calibration information generation unit 140 may use the startup-time concentration Ds, acquired during the period P1, for generating calibration information, on condition that an external temperature of the mobile body 200 at startup of the component detection unit 110 is within a preset temperature range. At startup of the component detection unit 110, it may be possible to estimate that the temperature in the operator’s cabin 240 is approximately equal to an external temperature. Therefore, by using an external temperature in place of a temperature of the component detection unit 110 in the aforementioned process, influence of variation in a temperature of the component detection unit 110 at a time of measuring the startup-time concentration Ds can be reduced. The temperature range may allow the component detection unit 110 to detect the startup-time concentration Ds with high accuracy. The temperature range may be, for example, 10°C or higher and 30°C or lower. In another example, the calibration information generation unit 140 may correct each of the startup-time concentrations Ds based on an external temperature at a time when the startup-time concentration Ds was measured. The mobile body 200 may be provided with a temperature sensor which detects an external temperature.

The calibration information generation unit 140 may select the startup-time concentration Ds (or startup-time detection information) to be used for generating calibration information, based on a difference between a temperature of the component detection unit 110 at startup of the component detection unit 110 and an external temperature of the mobile body 200 (referred to as an outside air temperature). When an operator or the like is in the operator’s cabin 240, the temperature in the operator’s cabin 240 is adjusted by an air conditioner or the like to a temperature different from an outside air temperature. Meanwhile, when an operator or the like leaves the operator’s cabin 240 before the time T1 and an air conditioner stops, the temperature in the operator’s cabin 240 gradually gets closer to an outside air temperature. Therefore, the longer the time between an operator or the like leaving the operator’s cabin 240 before the time T1 and startup of equipment such as the component detection unit 110 at the time T2, the closer the temperature in the operator’s cabin 240 gets to an outside air temperature, and the smaller the temperature difference becomes. Therefore, it can be estimated that the smaller the temperature difference, the closer the carbon dioxide concentration in the operator’s cabin 240 is to the outside air carbon dioxide concentration Da. The calibration information generation unit 140 may use the startup-time concentration Ds, acquired during the period P1, for generating calibration information, on condition that a difference between a temperature of the component detection unit 110 at startup of the component detection unit 110 and an outside air temperature is equal to or smaller than a preset reference value.

The calibration information generation unit 140 may select the startup-time concentration Ds (or startup-time detection information) to be used for generating calibration information, based on an air pressure inside or outside the mobile body 200 at startup of the component detection unit 110. For example, for each of the periods P1, the calibration information generation unit 140 may use the startup-time concentration Ds, acquired during the period P1, for generating calibration information, on condition that an air pressure inside or outside the mobile body 200 at startup of the component detection unit 110 is within a preset air pressure range. At startup of the component detection unit 110, it may be possible to estimate that an air pressure inside the operator’s cabin 240 is approximately equal to an air pressure outside. Therefore, either an air pressure inside or outside the mobile body 200 may be used. While a gas concentration to be measured does not fluctuate due to air pressure, a concentration indication value indicated by the component detection unit 110 may fluctuate due to a nature of a measurement mechanism. Therefore, by selecting the startup-time concentration Ds (or startup-time detection information) on condition that air pressure is within a certain range, influence of variations in the air pressure at a time of measuring the startup-time concentration Ds can be reduced. The air pressure range may be within a ±2% range, may be within a ±1% range of standard atmospheric pressure, or may be within another range. In another example, the calibration information generation unit 140 may correct each of the startup-time concentrations Ds based on an air pressure at a time when the startup-time concentration Ds was measured. The mobile body 200 may be provided with an air pressure sensor which detects an air pressure inside or outside it.

The calibration information generation unit 140 may select the startup-time concentration Ds (or startup-time detection information) to be used for generating calibration information, based on weather information regarding weather at startup of the component detection unit 110. Weather information may be acquired at startup of the component detection unit 110. The calibration information generation unit 140 may acquire weather information such as weather forecasts via a network such as the Internet. Weather information may be information indicating weather at the most recent time among information acquired at startup of the component detection unit 110. Weather information may be information indicating whether it is sunny or not sunny. Weather information may be information indicating at least any of sunny, rainy, or cloudy conditions. For example, for each of the periods P1, the calibration information generation unit 140 may use the startup-time concentration Ds, acquired during the period P1, for generating calibration information, on condition that weather information at startup of the component detection unit 110 satisfies a preset weather condition. The weather condition may be, for example, a “sunny” condition. Alternatively, the weather condition may be most typical weather at a current location of the mobile body 200. For example, in a location where a “cloudy” condition predominates throughout a year, the weather condition may be a “cloudy” condition. Since air pressure and humidity, which can be causes of variation in an indication value of a gas sensor, are correlated with weather, it is possible to improve accuracy of calibration information by limiting a weather condition.

A carbon dioxide concentration in the operator’s cabin 240 may decrease due to events such as ventilation. In the present example, the operator’s cabin 240 of the mobile body 200 includes one or more windows 250 which are openable and closable. In the example of FIG. 3, at a time T3, any one of the windows 250 is controlled to be in an open state, allowing air to flow between inside and outside of the operator’s cabin 240. In this case, a carbon dioxide concentration in the operator’s cabin 240 gradually gets closer to the outside air carbon dioxide concentration Da.

The calibration information generation unit 140 acquires opening and closing information of the window 250 at a time when the component detection unit 110 obtains detection information of carbon dioxide through detection, and, based on the opening and closing information, selects detection information of carbon dioxide to be used for generating calibration information. Opening and closing information may be acquired from the control unit which controls opening and closing of the window 250. When opening and closing of the window 250 is controlled by an electric signal, opening and closing information may be generated based on the electric signal. When opening and closing of the window 250 is manually controlled, the mobile body 200 may include a position sensor which detects opening and closing information by detecting an edge position or the like of the window 250.

For example, the calibration information generation unit 140 may generate calibration information by selecting detection information of carbon dioxide detected by the component detection unit 110 in a state where an opening degree of the window 250 is equal to or greater than a predetermined value. The opening degree of the window 250 may be a ratio of an opening area of the window 250 in a state for which the opening degree is to be calculated, to the opening area in a fully open state in which the window 250 connects the operator’s cabin 240 to the outside. Furthermore, the calibration information generation unit 140 may determine that the opening degree of the window 250 is equal to or greater than a predetermined value when an air conditioning system of the mobile body 200 is set to an outside air intake mode instead of an interior air recirculation mode. The calibration information generation unit 140 may generate calibration information by selecting a concentration of carbon dioxide (referred to as a post-window opening concentration Dw) measured after a predetermined period P2 has elapsed since the time T3 at which the opening degree of the window 250 becomes equal to or greater than a predetermined value. The longer the period P2 is, the closer the post-window opening concentration Dw gets to the outside air carbon dioxide concentration Da. The component detection unit 110 may measure the post-window opening concentration Dw of carbon dioxide after the period P2 has elapsed since the time T3. Similarly to the period P1, the calibration information generation unit 140 may estimate the outside air concentration Da from changes over time in carbon dioxide obtained through multiple detections within the period P2, and may generate calibration information based on the estimated outside air concentration Da.

The calibration information generation unit 140 may adjust a length of the period P2 based on the opening degree of the window 250. For example, the period P2 may be adjusted to be shorter as the opening degree of the window 250 is larger. The calibration information generation unit 140 may adjust the length of the period P2 based on a moving speed of the mobile body 200 at the time T3. The calibration information generation unit 140 may adjust the period P2 to be shorter as the moving speed of the mobile body 200 is faster. The calibration information generation unit 140 may generate calibration information by selecting a carbon dioxide concentration measured in a state where the moving speed of the mobile body 200 is equal to or greater than a set value and the opening degree of the window 250 is equal to or greater than a set value. The period P2 may be one minute or longer, may be 2 minutes or longer, or may be 10 minutes or longer.

In the example of FIG. 3 the window 250 is closed at the time T4, and carbon dioxide concentration gradually increases. In the example of FIG. 3 , energy supply to the power unit 220 is stopped at the time T5. The component detection unit 110 may detect a carbon dioxide concentration and an alcohol concentration at regular intervals between the time T2 and the time T5. The component detection unit 110 may detect a carbon dioxide concentration and an alcohol concentration at predetermined timings between the time T2 and the time T5. At time T6, an operator or the like leaves the operator’s cabin 240. As a result, a carbon dioxide concentration in the operator’s cabin 240 gradually decreases.

In a case where the power unit 220 is driven by electric power to generate power for moving the mobile body 200, the electric power storage unit 230 may be charged by a user or the like after the power unit 220 is stopped. In this case, the component detection unit 110 may receive electric power from the electric power storage unit 230 or a charging means, and detect a carbon dioxide concentration. The calibration information generation unit 140 may generate calibration information by using a stoppage-time concentration Dp (or stoppage-time detection information) of carbon dioxide concentration detected in a state where the power unit 220 of the mobile body 200 is stopped and the electric power storage unit 230 is charged. In the present specification, detection information corresponding to the stoppage-time concentration is referred to as stoppage-time detection information.

The calibration information generation unit 140 may generate calibration information by using the stoppage-time concentration Dp detected after a predetermined period P3 has elapsed since the time T5. The calibration information generation unit 140 may adjust the period P3 according to a carbon dioxide concentration in the operator’s cabin 240 at the time T5. For example, the higher the carbon dioxide concentration in the operator’s cabin 240 at the time T5, the longer the period P3 may be. The period P3 may be an hour or longer or may be 2 hours or longer.

The component detection unit 110 may or may not detect a carbon dioxide concentration during the period P3. When the component detection unit 110 periodically detects a carbon dioxide concentration during the period P3, the calibration information generation unit 140 may generate calibration information by using the stoppage-time concentration Dp after a fluctuation amount of carbon dioxide concentration per unit time becomes equal to or less than a set value. When the component detection unit 110 does not detect a carbon dioxide concentration during the period P3, power consumption can be reduced. Similarly to the period P1, the calibration information generation unit 140 may estimate the outside air concentration Da from changes over time in carbon dioxide obtained through multiple detections within the period P3, and may generate calibration information based on the estimated outside air concentration Da.

The calibration information generation unit 140 may generate calibration information by using one or more types of the startup-time concentration Ds, the post-window opening concentration Dw, and the stoppage-time concentration Dp. The calibration information generation unit 140 may generate calibration information by using the plurality of the startup-time concentrations Ds, may generate calibration information by using a plurality of the post-window opening concentrations Dw, and may generate calibration information by using a plurality of the stoppage-time concentrations Dp. Further, in the present specification, detection information corresponding to the stoppage-time concentration may be referred to as the stoppage-time detection information.

The calibration information generation unit 140 may acquire opening and closing history information indicating an opening and closing history of the door 260, and based on the opening and closing history information, may select detection information of carbon dioxide to be used for generating calibration information. The calibration information generation unit 140 may calculate a number of people present in the operator’s cabin 240 at a time when a carbon dioxide concentration was detected, based on the opening and closing history information. For example, if the mobile body 200 has a plurality of seats, and the door 260 corresponding to each seat is provided, the number of people can be estimated based on the opening and closing history information of each of the doors 260. For example, a number of the doors 260 opened and closed before and after startup of the power unit 220 may be regarded as the number of people who have entered the operator’s cabin 240. The calibration information generation unit 140 may generate calibration information by using a corresponding carbon dioxide concentration when the number of people is equal to or less than a reference value. The reference value may be, for example, one person. The greater the number of people who have entered the operator’s cabin 240, the more likely the startup-time concentration Ds becomes higher than the outside air carbon dioxide concentration Da. By selecting a concentration of carbon dioxide for generating calibration information based on the opening and closing history information, the startup-time concentration Ds, which is close to the outside air carbon dioxide concentration Da, can be extracted.

The calibration information generation unit 140 may use, in place of the opening and closing history information of the door 260, seat belt fastening and unfastening history information, seat occupancy sensor information, or millimeter-wave sensor information for each seat. The calibration information generation unit 140 may calculate the number of people present in the operator’s cabin 240 based on the seat belt fastening and unfastening history information, seat occupancy sensor information, or millimeter-wave sensor information for each seat.

The calibration information generation unit 140 may acquire location information of the mobile body 200 at a time when the component detection unit 110 obtains detection information of carbon dioxide through detection, and, based on the location information, may select detection information of carbon dioxide to be used for generating calibration information. For example, the calibration information generation unit 140 may select detection information of carbon dioxide obtained when the mobile body 200 was present within a preset range. The set range may be, for example, a range in which it can be determined that the mobile body 200 is parked at a residence of an operator. By generating calibration information from detection information selected based on location information of the mobile body 200, it is possible to eliminate influence of a location of the mobile body 200 on a concentration of carbon dioxide, thereby generating calibration information. For example, a carbon dioxide concentration in an underground parking lot or the like may differ from the outside air carbon dioxide concentration Da. For example, since detection information of carbon dioxide concentration obtained when the mobile body 200 is present at a certain location is used in the present example, fluctuations in a carbon dioxide concentration due to a location of the mobile body 200 can be decreased, and thus, calibration information can be generated with high accuracy.

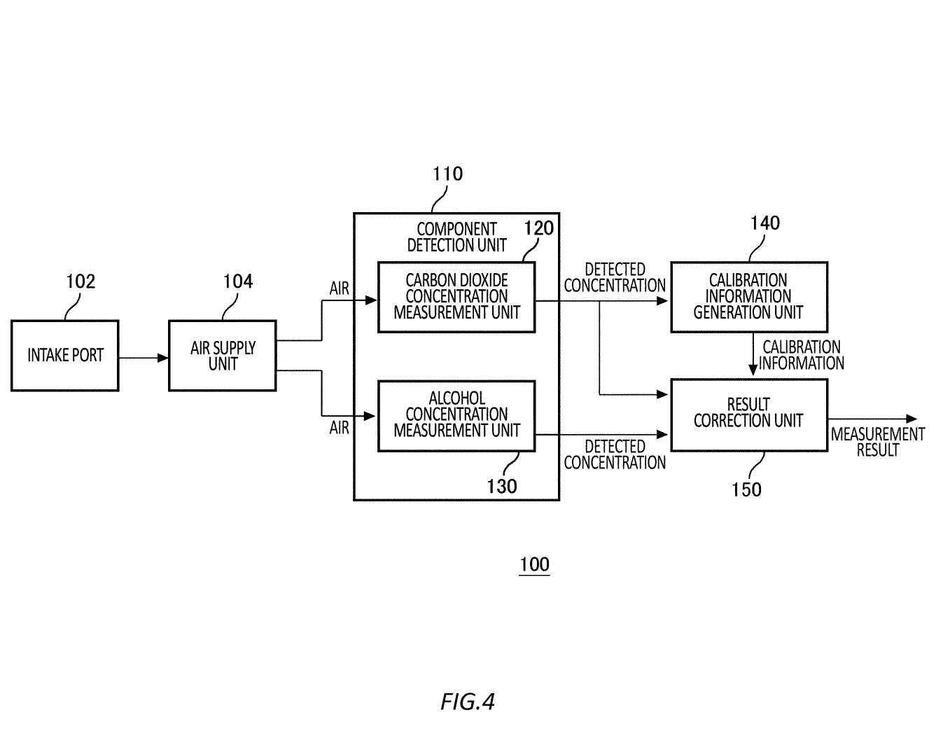

FIG. 4 is a block diagram showing another example configuration of the exhaled breath detection apparatus 100. The exhaled breath detection apparatus 100 in the present example further includes an air supply unit 104, in addition to the example shown in FIG. 2 . Other structures are the same as those in the example of FIG. 2. The calibration information generation unit 140 in the example of FIG. 2 , generated calibration information from a plurality of pieces of detection information of carbon dioxide. The calibration information generation unit 140 in the present example may generate calibration information from a piece of detection information of carbon dioxide or may generate calibration information from a plurality of pieces of detection information of carbon dioxide, as in the example of FIG. 2.

The air supply unit 104 is operable in a first state and a second state different from the first state in at least one of an airflow rate or an airflow direction, which supplies air from the operator’s cabin 240 to the component detection unit 110. An airflow rate in the present example is a flow rate of air per unit time. The air supply unit 104 is, for example, a fan which moves air by rotating a blade member, but a structure of the air supply unit 104 is not limited thereto. The air supply unit 104 blows air from the operator’s cabin 240 in a direction toward the component detection unit 110 in at least one of the first state or the second state.

The air supply unit 104 in the first state blows a smaller volume of air in a direction toward the component detection unit 110 than the air supply unit 104 in the second state. A volume of air directed toward the component detection unit 110 can be adjusted by at least one of an airflow rate or an airflow direction of the air supply unit 104.

The calibration information generation unit 140 generates calibration information based on detection information of carbon dioxide detected by the component detection unit 110 when the air supply unit 104 is in the first state. In the first state, air from the operator’s cabin 240 is less likely to pass through the intake port 102 and be detected by the component detection unit 110 than in the second state. Therefore, the component detection unit 110 in the first state can measure a carbon dioxide concentration in a state where influence of exhaled breath of an operator or the like is reduced. Accordingly, by using detection information of carbon dioxide in the first state, calibration information can be generated with high accuracy.

The result correction unit 150 corrects a detection result of alcohol based on a calibrated concentration of carbon dioxide obtained by calibrating, based on the calibration information, detection information of the carbon dioxide detected by the component detection unit 110 when the air supply unit 104 is in the second state. A method for correcting a detection result of alcohol based on a calibrated concentration of carbon dioxide is the same as in the example of FIG. 2, When the air supply unit 104 is in the second state, air from the operator’s cabin 240 is more likely to pass through the intake port 102 and be detected by the component detection unit 110. This makes it easier to measure exhaled breath of an operator.

As in the present example, by generating calibration information from detection information of carbon dioxide when the air supply unit 104 is in the first state, influence of an exhaled breath of an operator can be reduced, and thus calibration information can be generated with high accuracy. Furthermore, by determining a detection result of alcohol from detection information of carbon dioxide and alcohol when the air supply unit 104 is in the second state, a detection result can be determined with high accuracy.

The calibration information generation unit 140 may acquire state history information indicating which state the air supply unit 104 was in when the component detection unit 110 obtained detection information of carbon dioxide through detection. The calibration information generation unit 140 may select detection information of carbon dioxide for generating calibration information based on the state history information. The calibration information generation unit 140 may select detection information of carbon dioxide for generating calibration information, further based on each condition described in FIG. 3. For example, in both the present example and the example described in FIG. 3, calibration information may be generated by extracting detection information of carbon dioxide that should be selected for generating calibration information.

The calibration information generation unit 140 may control the air supply unit 104 to be in the first state at a timing when detection information of carbon dioxide to be used for generating calibration information should be measured. This timing is the same as a detection timing at which detection information of carbon dioxide to be used for generating calibration information is detected, for example, in the example described in FIG. 3 . By combining the present example and the example in FIG. 3, calibration information can be generated with higher accuracy.

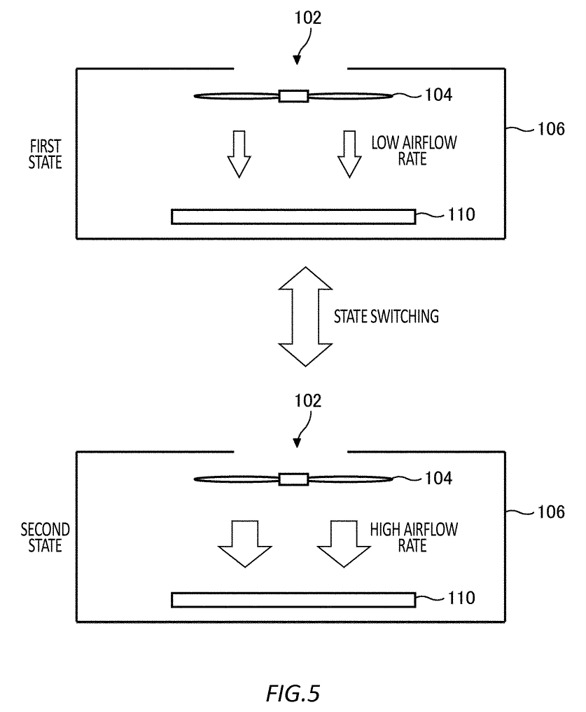

FIG. 5, is a schematic diagram illustrating a state of the air supply unit 104. In FIG. 5 , the intake port 102, the air supply unit 104, the component detection unit 110, and a detection housing 106 are illustrated. The detection housing 106 is a box-shaped housing which accommodates the component detection unit 110. The detection housing 106 is formed of, for example, resin, metal, or the like. The intake port 102 is provided in the detection housing 106. The intake port 102 is an opening through which air can flow between inside and outside of the detection housing 106.

The air supply unit 104 is disposed opposite the intake port 102. The air supply unit 104 may be provided inside the detection housing 106 or may be provided outside thereof. The air supply unit 104 in the present example is provided between the intake port 102 and the component detection unit 110. When the air supply unit 104 is driven, it draws air from the operator’s cabin 240 through the intake port 102 and blows the air toward the component detection unit 110.

The air supply unit 104 may operate so that a volume of air drawn into the exhaled breath detection apparatus 100 (or the detection housing 106) in the second state is greater than a volume of air drawn into the exhaled breath detection apparatus 100 (or the detection housing 106) in the first state. The volume of air may be a volume per unit time (liters per second). A volume of air drawn into the detection housing 106 can be controlled by an airflow rate of the air supply unit 104. In the present example, an airflow rate (liters per second) of the air supply unit 104 varies between the first state and the second state. An airflow direction of the air supply unit 104 may or may not vary between the first state and the second state. In the present example, an airflow rate of the air supply unit 104 in the first state is lower than an airflow rate of the air supply unit 104 in the second state. An airflow rate in the first state may be half or less, may be one-fourth or less, of an airflow rate in the second state, or may be zero. An amount of rotation of a fan of the air supply unit 104 in the first state may be less than an amount of rotation of the fan in the second state. The amount of rotation of the fan in the first state may be half or less, may be one-fourth or less, of the amount of rotation of the fan in the second state, or may be zero. The amount of rotation of the fan is a number of rotations per unit time.

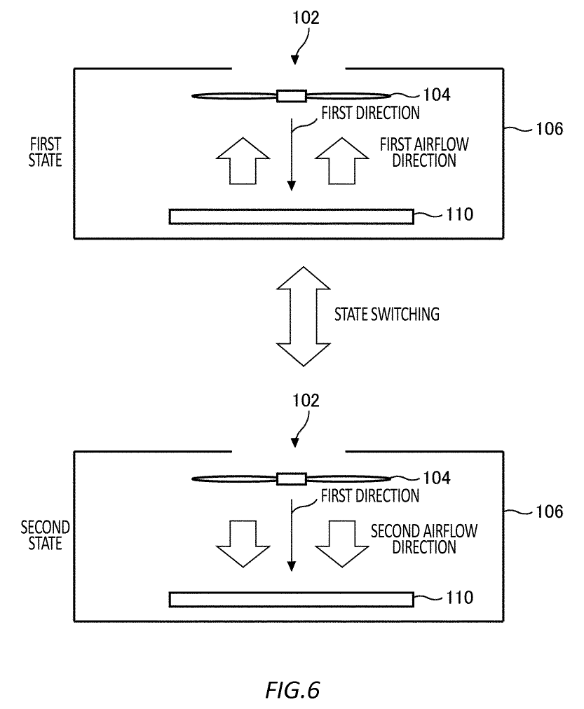

FIG. 6 is a schematic diagram illustrating the state of the air supply unit 104. Structures of the air supply unit 104 or the like are the same as those in the example of FIG. 5. In the present example, an airflow direction of the air supply unit 104 varies between the first state and the second state. An airflow rate of the air supply unit 104 may not vary between the first state and the second state, or may vary as in the example of FIG. 5.

In the present example, a direction from the air supply unit 104 toward the component detection unit 110 is defined as a first direction. For example, the first direction may be defined as a direction from a center point of an air-blowing surface (a surface through which air passes) of the air supply unit 104 toward a center point of a detection surface (a surface sensitive to carbon dioxide) of the component detection unit 110. A difference between the first direction from the air supply unit 104 toward the component detection unit 110 and an airflow direction (this difference is referred to as a directional difference in the present specification) is greater in the air supply unit 104 in the first state than in the air supply unit 104 in the second state.

An airflow direction refers to a direction in which the largest volume of air is blown from the air supply unit 104. For example, an airflow direction is parallel to a direction perpendicular to a rotating surface of the fan of the air supply unit 104. In the example of FIG. 6 , a directional difference in the first state is 180 degrees, and a directional difference in the second state is 0 degrees. For example, in the first state and the second state, by rotating the fan of the air supply unit 104 in a reverse direction, airflow directions in the first state and the second state can be reversed. The directional difference in the first state may be other than 180 degrees. The directional difference in the first state may be 90 degrees or more and 180 degrees or less. The directional difference in the second state may be other than 0 degrees. The directional difference in the second state may be 0 degrees or more and 45 degrees or less. For example, the directional difference may be controlled by controlling a tilt of a rotation axis or the air-blowing surface of the air supply unit 104.

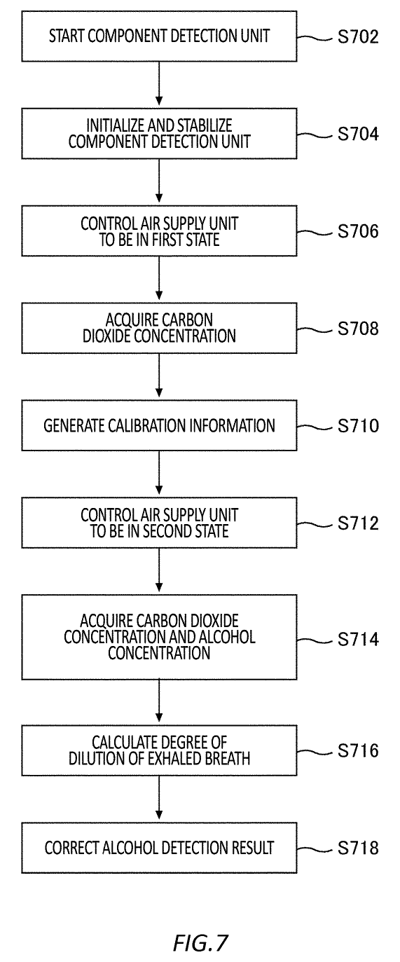

FIG. 7 is a flowchart illustrating an example operation of the exhaled breath detection apparatus 100 illustrated in FIG. 4. In the example of FIG. 7, a part of operation of the exhaled breath detection apparatus 100 is described. The exhaled breath detection apparatus 100 may perform each operation described in the present specification. The air supply unit 104 in the present example transitions to the first state after startup of the mobile body 200, and transitions to the second state after the first state.

In step S702, the component detection unit 110 is started. Processing in step S702 is the same as processing at the time T2 in FIG. 3 . Startup of the component detection unit 110 may occur simultaneously with startup of the mobile body 200.

In step S704, the component detection unit 110 is initialized, and measurement becomes stable. In step S704, from the time T2 until a set period elapses, the air supply unit 104 and the calibration information generation unit 140 may remain in standby. This period is shorter than the period P1 described in FIG. 3.

In step S706, the air supply unit 104 is controlled to be in the first state. The calibration information generation unit 140 may control the state of the air supply unit 104, or the exhaled breath detection apparatus 100 may include the control unit which controls the state of the air supply unit 104. Processing in step S706 is performed, for example, within the period P1.

In step S708, the component detection unit 110 obtains detection information of carbon dioxide through detection, and the calibration information generation unit 140 acquires the detection information. Processing in which the component detection unit 110 obtains detection information of carbon dioxide through detection is performed, for example, within the period P1.

In step S710, the calibration information generation unit 140 generates calibration information based on the detection information acquired in step S708. In step S710, when current detection information updates a minimum value among detection information acquired through multiple detections in step S708 within a past set period (that is, when the corresponding concentration updates the minimum value), the calibration information may be updated. If the current detection information does not update the minimum value, the calibration information generation unit 140 may use previously generated calibration information.

In step S712, the air supply unit 104 is controlled to be in the second state. Processing in step S712 may be performed within the period P1, or may be performed after the period P1 has elapsed. Step S712 may be performed before movement of the mobile body 200 is started.

In step S714, the component detection unit 110 obtains detection information of carbon dioxide and alcohol through detection. Processing in step S714 may be performed within the period P1, or may be performed after the period P1 has elapsed. Step S714 may be performed before the movement of the mobile body 200 is started.

In step S716, the result correction unit 150 calculates a degree of dilution of exhaled breath of an operator based on detection information of carbon dioxide and calibration information. Processing in step S716 may be performed within the period P1, or may be performed after the period P1 has elapsed. Step S716 may be performed before the movement of the mobile body 200 is started.

In step S718, the result correction unit 150 corrects an alcohol detection result. As described above, the result correction unit 150 may correct a concentration of alcohol derived from detection information of alcohol, based on a degree of dilution of exhaled breath. Processing in step S718 may be performed within the period P1, or may be performed after the period P1 has elapsed. Step S718 may be performed before the movement of the mobile body 200 is started. By performing such processing, calibration information for a concentration of carbon dioxide can be generated with high accuracy. Furthermore, a detection result of alcohol can be corrected with high accuracy.

In each example described in the present specification, the operator’s cabin 240 may have a plurality of seats. When comparing distances between the component detection unit 110 (or the intake port 102 when the intake port 102 is provided) and each seat, a distance between a seat provided with equipment for operation and the component detection unit 110 (or the intake port 102 when the intake port 102 is provided) may be the shortest. The distance may be a distance to a top edge of the seat. The distance may be equal to or less than 1 m, or may be equal to or less than 50 cm. For example, in a case where the mobile body 200 is an automobile, the component detection unit 110 (or the intake port 102 when the intake port 102 is provided) may be disposed near an instrument panel or a steering wheel. By disposing the component detection unit 110 (or the intake port 102 when the intake port 102 is provided) near the instrument panel or the steering wheel, exhaled breath of an operator can be efficiently drawn in, thereby enabling improved detection performance of the component detection unit 110.

A timing of acquiring detection information of carbon dioxide to be used for generating calibration information may be set by an operator or the like. Alternatively, it may be controlled by an application installed in a mobile terminal of an operator or the like.

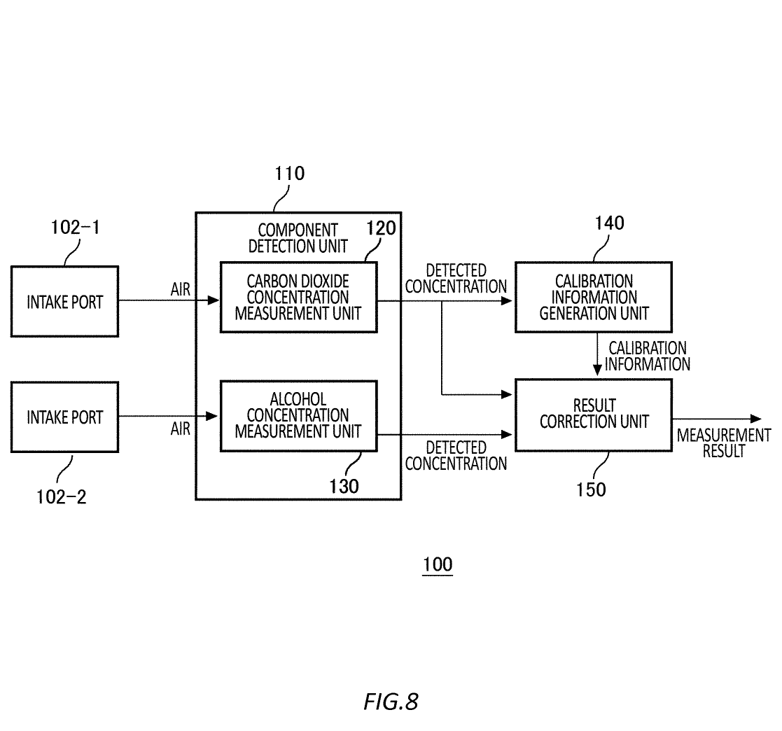

FIG. 8 shows another example configuration of the exhaled breath detection apparatus 100. The component detection unit 110 in the present example includes the carbon dioxide concentration measurement unit 120 and the alcohol concentration measurement unit 130, which are disposed at different locations. The component detection unit 110 in the present example is conceptually configured to include a plurality of structures disposed at different locations. In the present example, the intake port 102, the air supply unit 104 (see FIG. 4), and the detection housing 106 (see FIG. 5) may be provided respectively for the carbon dioxide concentration measurement unit 120 and the alcohol concentration measurement unit 130. Other functions and structures are the same as those in any of the examples described in FIGS. 1 through 7. As an example, one of the carbon dioxide concentration measurement unit 120 or the alcohol concentration measurement unit 130 may be provided on the door 260, and the other may be provided on equipment for operation such as a steering wheel.

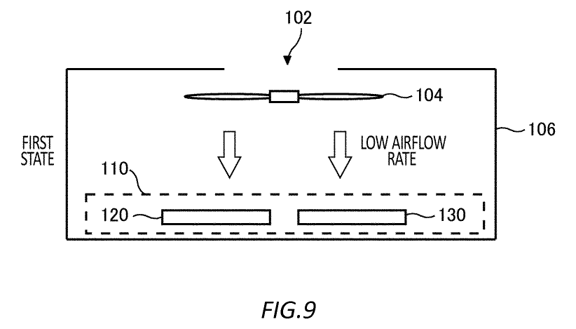

FIG. 9 shows an example configuration of the intake port 102, the air supply unit 104, the detection housing 106, and the component detection unit 110. In the present example, the airflow rate in the first state is shown. Airflow rates in each state are the same as those in the examples of FIG. 5 or FIG. 6. Configurations of the intake port 102, the air supply unit 104, the detection housing 106, and the component detection unit 110 in the present example are the same as those in the examples described in FIG. 5 or FIG. 6. In FIG. 9, the carbon dioxide concentration measurement unit 120 and the alcohol concentration measurement unit 130 of the component detection unit 110 are explicitly shown.

The intake port 102, the air supply unit 104, and the detection housing 106 are commonly provided for the carbon dioxide concentration measurement unit 120 and the alcohol concentration measurement unit 130 in the present example. That is, the carbon dioxide concentration measurement unit 120 and the alcohol concentration measurement unit 130 in the present example measure air from a same location.

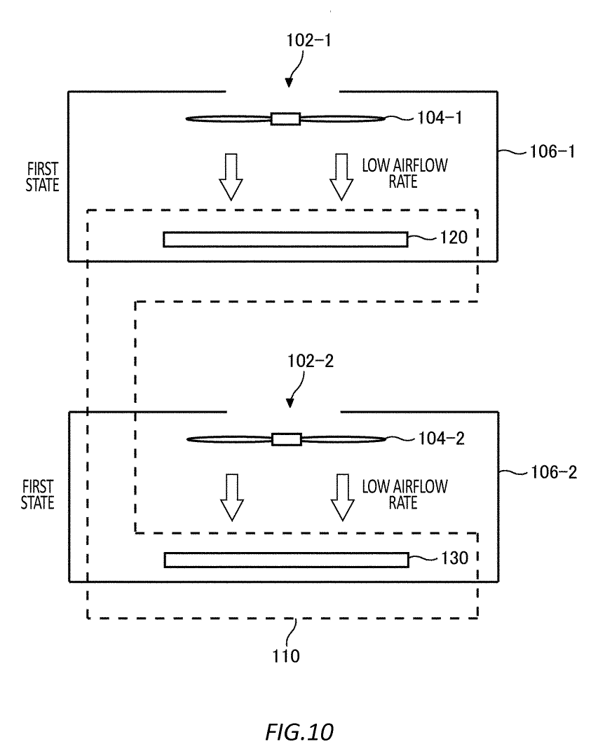

FIG. 10, shows another example configuration of the intake port 102, the air supply unit 104, the detection housing 106, and the component detection unit 110. The present example corresponds to the configuration in Fig.8. In the present example as well, the airflow rate in the first state is shown. Airflow rates in each state are the same as those in the examples of FIG. 5 or FIG. 6.

The intake port 102, the air supply unit 104, and the detection housing 106 are respectively provided for the carbon dioxide concentration measurement unit 120 and the alcohol concentration measurement unit 130 in the present example. The carbon dioxide concentration measurement unit 120 and the alcohol concentration measurement unit 130 in the present example can measure air from different locations.

The calibration information generation unit 140 and the result correction unit 150 described in FIG. 1 through FIG. 10 may be implemented by installing programs on one or more computers. These programs may be recorded on a computer-readable medium.

Various embodiments of the present invention may be described with reference to flowcharts and block diagrams, where blocks may represent (1) stages of processes in which operations are executed or (2) sections of apparatuses responsible for executing operations. Certain stages and sections may be implemented by a dedicated circuit, a programmable circuit supplied together with computer-readable instructions stored on computer-readable media, and/or processors supplied together with computer-readable instructions stored on computer-readable media. The dedicated circuit may include digital and/or analog hardware circuits, and may include integrated circuits (IC) and/or discrete circuits. The programmable circuit may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by a suitable device, and as a result, the computer-readable medium having instructions stored thereon includes a product including instructions that can be executed in order to create means for executing operations specified in the flowcharts or block diagrams. Examples of the computer-readable medium may include an electronic storage medium, a magnetic storage medium, an optical storage medium, an electromagnetic storage medium, a semiconductor storage medium, or the like. More specific examples of the computer-readable medium may include a FLOPPY (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disk (DVD), a BLU-RAY (registered trademark) disk, a memory stick, an integrated circuit card, or the like.

The computer-readable instruction may include: an assembler instruction, an instruction-set-architecture (ISA) instruction; a machine instruction; a machine dependent instruction; a microcode; a firmware instruction; state-setting data; or either a source code or an object code written in any combination of one or more programming languages, including an object oriented programming language such as SMALLTALK (registered trademark), JAVA (registered trademark), C++, or the like, and a conventional procedural programming language such as a "C" programming language or a similar programming language.

The computer-readable instructions may be provided for a processor or programmable circuit of a general-purpose computer, special-purpose computer, or other programmable data processing apparatuses such as a computer locally or via a wide area network (WAN) such as a local area network (LAN), the Internet, or the like, and execute the computer-readable instructions in order to create means for executing the operations designated in flowcharts or block diagrams. Here, a computer may be a PC, or a personal computer, a tablet computer, a smartphone, a workstation, a server computer, a general-purpose computer, a special-purpose computer, or the like, or may be a computer system in which a plurality of computers are connected to each other. Such computer system in which the plurality of computers are connected to each other is also called a distributed computing system, and is a computer in a broad sense. In the distributed computing system, the plurality of computers collectively execute the program by each of the plurality of computers executing a part of the program, and passing data during the execution of the program among the computers as needed.

Examples of the processor include a computer processor, a central processing unit (CPU), a processing unit, a microprocessor, a digital signal processor, a controller, a microcontroller, or the like. The computer may include one processor or a plurality of processors. In a multiprocessor system including a plurality of processors, the plurality of processors collectively execute a program by each of the processors executing a part of the program, and passing data during the execution of the program among the processors as needed. For example, in execution of multiple tasks, each of the plurality of processors may execute a portion of each task pieces by pieces by performing task-switching for each time slice. In this case, which portion of one program each processor is responsible for executing dynamically changes. Moreover, which portion of the program each of the plurality of processor is responsible for executing may be determined statically by multiprocessor-aware programming.

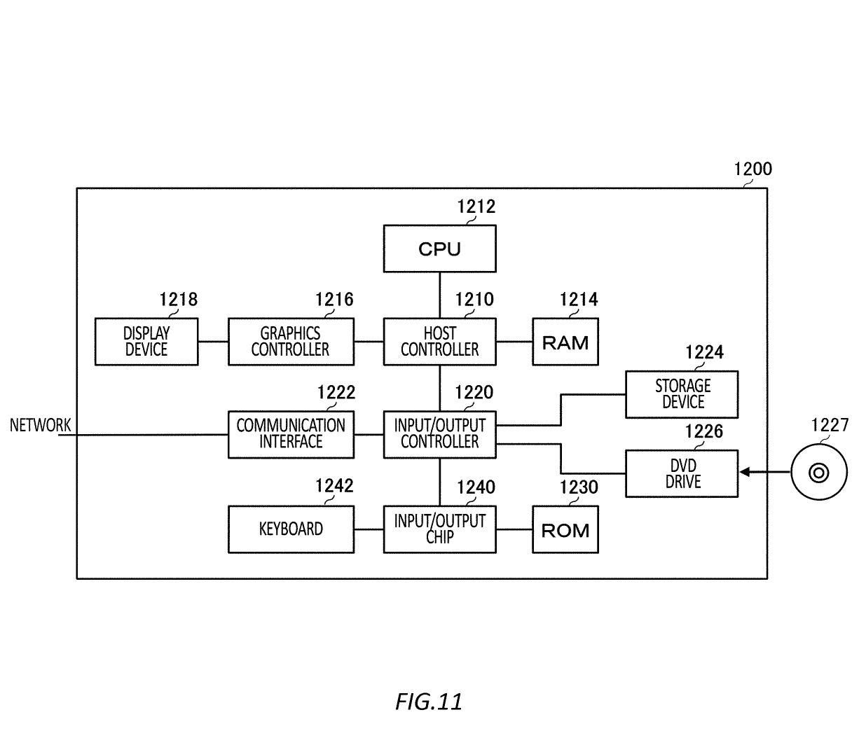

FIG. 11 shows an example of a computer 1200 in which a plurality of aspects of the present invention may be entirely or partially embodied. A program that is installed in the computer 1200 may cause the computer 1200 to function as operations associated with an apparatus according to the embodiment of the present invention or one or more sections in the apparatus, or may cause the computer 1200 to execute the operation or the one or more sections, and/or may cause the computer 1200 to execute processes according to the embodiment of the present invention or stages of the processes. Such a program may be executed by a CPU 1212 in order to cause the computer 1200 to execute particular operations associated with some or all of the blocks of flowcharts and block diagrams described herein.

The computer 1200 according to the present embodiment includes a CPU 1212, a RAM 1214, a graphics controller 1216, and a display device 1218, which are mutually connected by a host controller 1210. The computer 1200 also includes a communication interface 1222, a storage device 1224 such as a hard disk drive, input/output units such as a DVD-ROM drive 1226 and an IC card drive, which are connected to the host controller 1210 via an input/output controller 1220. The computer also includes legacy input/output units such as an ROM 1230 and a keyboard 1242, which are connected to the input/output controller 1220 via an input/output chip 1240.

The CPU 1212 operates according to programs stored in the ROM 1230 and the RAM 1214, thereby controlling each unit. The graphics controller 1216 acquires image data generated by the CPU 1212 on a frame buffer or the like provided in the RAM 1214 or in itself, and causes the image data to be displayed on a display device 1218.

The communication interface 1222 communicates with other electronic devices via a network. The storage device 1224 stores a program and data used by the CPU 1212 in the computer 1200. The DVD-ROM drive 1226 reads a program or data from a DVD-ROM 1227 and provides the program or data to the storage device 1224 via the RAM 1214. The IC card drive reads the programs and the data from the IC card, and/or writes the programs and the data to the IC card.

The ROM 1230 stores therein a boot program or the like that is executed by the computer 1200 at the time of activation, and/or a program which depends on the hardware of the computer 1200. The input/output chip 1240 may also connect various input/output units to the input/output controller 1220 via a parallel port, a serial port, a keyboard port, a mouse port, or the like.

Programs are provided by a computer-readable medium such as the DVD-ROM 1227 or the IC card. The programs are read from the computer-readable medium, are installed in the storage device 1224, the RAM 1214, or the ROM 1230, which are also an example of the computer-readable medium, and are executed by the CPU 1212. Information processing written in these programs is read by the computer 1200, and provides cooperation between the programs and the various types of hardware resources described above. A device or a method may be configured by realizing the operation or processing of information according to the use of the computer 1200.

For example, when communication is executed between the computer 1200 and an external device, the CPU 1212 may execute a communication program loaded onto the RAM 1214 to instruct communication processing to the communication interface 1222, based on the processing written in the communication program. Under the control of the CPU 1212, the communication interface 1222 reads transmission data stored in a transmission buffer processing region provided in a recording medium such as the RAM 1214, the storage device 1224, the DVD-ROM 1227, or the IC card, transmits the read transmission data to the network, or writes reception data received from the network in a reception buffer processing region or the like provided on the recording medium.

The CPU 1212 may cause all or a needed portion of the file or database stored in an external recording medium such as a storage device 1224, a DVD-ROM drive 1226 (a DVD-ROM 1227), an IC card, or the like to be read to the RAM 1214, and perform various types of processing on the data on the RAM 1214. The CPU 1212 then writes back the processed data to the external recording medium.

Various types of information such as various types of programs, data, tables, and databases may be stored in a recording medium and subjected to information processing. The CPU 1212 may execute various types of processing on the data read from the RAM 1214, which includes various types of operations, information processing, conditional judging, conditional branch, unconditional branch, search/replace of information, or the like, as described throughout the present disclosure and designated by an instruction sequence of programs, and writes the result back to the RAM 1214. In addition, the CPU 1212 may search for information in a file, a database, or the like in the recording medium. For example, when a plurality of entries, each having an attribute value of a first attribute associated with an attribute value of a second attribute, are stored in the recording medium, the CPU 1212 may retrieve, out of the plurality of entries, an entry with the attribute value of the first attribute specified that meets a condition, read the attribute value of the second attribute stored in this entry, and thereby acquiring the attribute value of the second attribute associated with the first attribute satisfying a predetermined condition.

The above-described program or software module may be stored in the computer-readable medium on the computer 1200 or near the computer 1200. In addition, a recording medium such as a hard disk or a RAM provided in a server system connected to a dedicated communication network or the Internet can be used as the computer-readable medium, thereby providing the program to the computer 1200 via the network.

While the present invention has been described so far by way of the embodiments, the technical scope of the present invention is not limited to the scope described in the above embodiments. It is apparent to persons skilled in the art that various alterations or improvements can be made to the above-described embodiments. It is also apparent from description of the claims that the embodiments to which such alterations or improvements are made may be included in the technical scope of the present invention. The present specification further discloses inventions pertaining to each of the following items.

### Item 1

A calibration information generation apparatus which generates calibration information in an exhaled breath detection apparatus provided in an operator’s cabin of a mobile body, the exhaled breath detection apparatus obtaining detection information, through detection, indicating a concentration of alcohol and a concentration of carbon dioxide contained in exhaled breath, and corrects a detection result of the alcohol based on a calibrated concentration of the carbon dioxide obtained through calibration with the calibration information, wherein the calibration information generation apparatus generates the calibration information for calibrating a concentration of the carbon dioxide, based on the detection information of the carbon dioxide obtained through multiple detections.

### Item 2

A non-transitory computer-readable medium having recorded thereon a program which, when executed by a computer, causes the computer to perform an operation comprising: generating calibration information for calibrating a concentration of carbon dioxide based on detection information of the carbon dioxide obtained through multiple detections by a component detection unit.

It should be noted that each process of the operations, procedures, steps, stages, and the like performed by the apparatus, system, program, and method shown in the claims, specification, and drawings can be implemented in any order as long as the order is not specifically and expressly indicated by “before”, “prior to”, or the like and as long as the output from a previous process is not used in a later process. Even if the operation flow is described by using phrases such as "first" or "next" for the sake of convenience in the claims, specification, and drawings, it does not necessarily mean that the process must be performed in this order.

What is claimed is:

1. An exhaled breath detection apparatus which detects alcohol contained in exhaled breath in an operator’s cabin of a mobile body, comprising:
a component detection unit which obtains, through detection, detection information indicating a concentration of the alcohol and a concentration of carbon dioxide contained in exhaled breath;
a calibration information generation unit which generates calibration information for calibrating a concentration of the carbon dioxide, based on the detection information of the carbon dioxide obtained through multiple detections by the component detection unit; and
a result correction unit which corrects a detection result of the alcohol based on a calibrated concentration of the carbon dioxide obtained through calibration with the calibration information.

2. The exhaled breath detection apparatus according to claim 1, wherein
the mobile body includes a power unit which generates power to move the mobile body, and the calibration information generation unit generates the calibration information by using startup-time detection information of the carbon dioxide detected within a preset period after startup of the power unit of the mobile body.

3. The exhaled breath detection apparatus according to claim 1, wherein
the calibration information generation unit generates the calibration information by using startup-time detection information of the carbon dioxide detected within a preset period after startup of the component detection unit.

4. The exhaled breath detection apparatus according to claim 3, wherein
the calibration information generation unit selects the startup-time detection information to be used for generating the calibration information, based on a temperature of the component detection unit at startup of the component detection unit.

5. The exhaled breath detection apparatus according to claim 3, wherein
the calibration information generation unit selects the startup-time detection information to be used for generating the calibration information, based on an external temperature of the mobile body at startup of the component detection unit.

6. The exhaled breath detection apparatus according to claim 3, wherein
the calibration information generation unit selects the startup-time detection information to be used for generating the calibration information, based on an air pressure inside or outside the mobile body at startup of the component detection unit.

7. The exhaled breath detection apparatus according to claim 3, wherein
the calibration information generation unit selects the startup-time detection information to be used for generating the calibration information, based on weather information regarding weather at startup of the component detection unit.

8. The exhaled breath detection apparatus according to claim 1, wherein
the mobile body includes a power unit which is driven by electric power to generate power for moving the mobile body, and an electric power storage unit which stores the electric power, and
the calibration information generation unit generates the calibration information by using stoppage-time detection information of the carbon dioxide detected in a state where the power unit of the mobile body is stopped and the electric power storage unit is charged.

9. The exhaled breath detection apparatus according to claim 1, wherein
the operator’s cabin of the mobile body includes a window which is openable and closable, and
the calibration information generation unit acquires opening and closing information of the window at a time when the component detection unit obtains the detection information of the carbon dioxide through detection, and based on the opening and closing information, selects the detection information of the carbon dioxide to be used for generating the calibration information.

10. The exhaled breath detection apparatus according to claim 1, wherein
the operator’s cabin of the mobile body includes a door which is openable and closable, and
the calibration information generation unit acquires opening and closing history information indicating an opening and closing history of the door, and based on the opening and closing history information, selects the detection information of the carbon dioxide to be used for generating the calibration information.

11. The exhaled breath detection apparatus according to claim 1, wherein
the calibration information generation unit acquires location information of the mobile body at a time when the component detection unit obtains the detection information of the carbon dioxide through detection, and, based on the location information, selects the detection information of the carbon dioxide to be used for generating the calibration information.

12. The exhaled breath detection apparatus according to claim 1, further comprising:
an air supply unit which is operable in a first state and a second state different from the first state in at least one of an airflow rate or an airflow direction, which supplies air from the operator’s cabin to the component detection unit, wherein
the calibration information generation unit generates the calibration information based on the detection information of the carbon dioxide detected by the component detection unit when the air supply unit is in the first state, and
the result correction unit corrects a detection result of the alcohol based on a calibrated concentration calibrated by calibrating, based on the calibration information, the detection information of the carbon dioxide detected by the component detection unit when the air supply unit is in the second state.

13. The exhaled breath detection apparatus according to claim 12, wherein
the air supply unit transitions to the first state after startup of the mobile body, and transitions to the second state after the first state.

14. The exhaled breath detection apparatus according to claim 12, wherein
the air supply unit in the first state has an airflow rate lower than an airflow rate of the air supply unit in the second state.

15. The exhaled breath detection apparatus according to claim 12, wherein
the air supply unit operates so that a volume of air drawn into the exhaled breath detection apparatus in the second state is greater than a volume of air drawn into the exhaled breath detection apparatus in the first state.

16. The exhaled breath detection apparatus according to claim 12, wherein
a difference between a direction from the air supply unit toward the component detection unit and the airflow direction is greater in the air supply unit in the first state than in the air supply unit in the second state.

17. An exhaled breath detection apparatus which detects alcohol contained in exhaled breath in an operator’s cabin of a mobile body, comprising:
a component detection unit which obtains, through detection, detection information indicating a concentration of the alcohol and a concentration of carbon dioxide contained in exhaled breath;
an air supply unit which is operable in a first state and a second state different from the first state in at least one of an airflow rate or an airflow direction, which supplies air from the operator’s cabin to the component detection unit;
a calibration information generation unit which generates calibration information for calibrating a concentration of the carbon dioxide, based on the detection information of the carbon dioxide detected by the component detection unit when the air supply unit is in the first state; and
a result correction unit which corrects a detection result of the alcohol based on a calibrated concentration obtained by calibrating, based on the calibration information, the detection information of the carbon dioxide detected by the component detection unit when the air supply unit is in the second state.

18. An exhaled breath detection apparatus which detects alcohol contained in exhaled breath in an operator’s cabin of a mobile body, comprising:
an alcohol concentration measurement unit which measures a concentration of the alcohol contained in exhaled breath;
a carbon dioxide concentration measurement unit which measures a concentration of carbon dioxide contained in exhaled breath;
a calibration information generation unit which generates calibration information for calibrating a concentration of the carbon dioxide, based on detection information of the carbon dioxide obtained through multiple detections by the carbon dioxide concentration measurement unit; and
a result correction unit which corrects a detection result of the alcohol based on a calibrated concentration of the carbon dioxide obtained through calibration with the calibration information.
